(19) United States
(12) Patent Application Publication
(10) Pub. No.: US 2025/0276113 A1
(43) Pub. Date: Sep. 4, 2025

(54) SHAFT FOR MEDICAL DEVICE, AND MEDICAL DEVICE

(71) Applicant: ASAHI INTECC CO., LTD., Seto-shi (JP)

(72) Inventors: Aoi OKAMURA, Seto-shi (JP); Kenta Tsuge, Seto-shi (JP)

(73) Assignee: ASAHI INTECC CO., LTD., Seto-shi (JP)

(21) Appl. No.: 19/210,073

(22) Filed: May 16, 2025

Related U.S. Application Data
(63) Continuation of application No. PCT/JP2023/042533, filed on Nov. 28, 2023.

(30) Foreign Application Priority Data
Nov. 30, 2022 (WO) PCT/JP2022/044156

(51) Int. Cl. A61L 31/14; A61M 25/09

(52) U.S. Cl. CPC A61L 31/14; A61M 25/09; A61L 2400/16; A61M 2025/09083; A61M 2025/09133; A61M 2025/09191

(57) ABSTRACT

A shaft for a medical device that has a ratio of a first Young's modulus measured by a bending test relative to a second Young's modulus measured by a tensile test of 1.2 or more and 1.7 or less.

### CROSS REFERENCE TO RELATED APPLICATIONS

This application is a continuation application of International Application No. PCT/JP2023/042533 filed Nov. 28, 2023, which claims priority to International Application No. PCT/JP2022/044156, filed Nov. 30, 2022. The contents of these applications are incorporated herein by reference in their entirety.

### TECHNICAL FIELD

This disclosure relates to a shaft for a medical device, and a medical device.

### BACKGROUND

Patent literature 1 discloses a multi-segment intraluminal guidewire including an elongated distal portion made of a first metallic material and an elongated proximal portion made of a second metallic material, in which the proximal portion is formed of the metallic material having a higher Young's modulus (i.e., higher rigidity) than the distal portion.

### CITATION LIST

### Patent Literature

Patent Literature 1: JP 2018-514259 W

### SUMMARY

### Technical Problem

In a shaft for a medical device, it is desirable to achieve a balance between tensile strength and torque transmission. However, in Patent literature 1, this balance is not taken into consideration at all. Note that such a problem is not limited to the vascular system but is common to all shafts for medical devices used in medical devices inserted into a body lumen, such as the one in the lymphatic system, biliary system, urinary system, respiratory tract system, digestive system, secretory gland, or reproductive organ.

This disclosure has been made to solve at least part of the above-mentioned problems and has an object to provide a shaft for a medical device with improved tensile strength and improved torque transmission.

### Solution to Problem

This disclosure has been made to solve at least part of the above-mentioned problems and can be achieved in the following aspects.

According to one aspect of this disclosure, a shaft for a medical device is provided. This shaft for a medical device has a ratio of a first Young's modulus measured by a bending test relative to a second Young's modulus measured by a tensile test of 1.2 or more and 1.7 or less.

Note that this configuration can be manifested in various aspects, for example, in the form of a shaft for a medical device, a medical device including the shaft for a medical device, a guidewire, methods for producing the same, and the like.

### BRIEF DESCRIPTION OF DRAWINGS

FIG. 1 is a cross-sectional view that illustrates a guidewire.

FIG. 2 is a perspective view of a core shaft.

FIG. 3 is a table showing results of various tests on a shaft for a medical device.

FIG. 4 is a schematic diagram illustrating a three-point bending test method.

FIG. 5 is a schematic diagram illustrating a tensile test method.

FIGS. 6A to 6C are schematic diagrams illustrating a restoration test method.

FIG. 7 is a schematic diagram illustrating a torque test method.

FIG. 8 is an enlarged cross-sectional view of a part of a tip side of a guidewire of a second embodiment.

FIG. 9 is an enlarged cross-sectional view of a part of the tip side of a guidewire of a third embodiment.

FIG. 10 is a schematic diagram illustrating a method for producing the shaft for a medical device.

### DETAILED DESCRIPTION

### First Embodiment

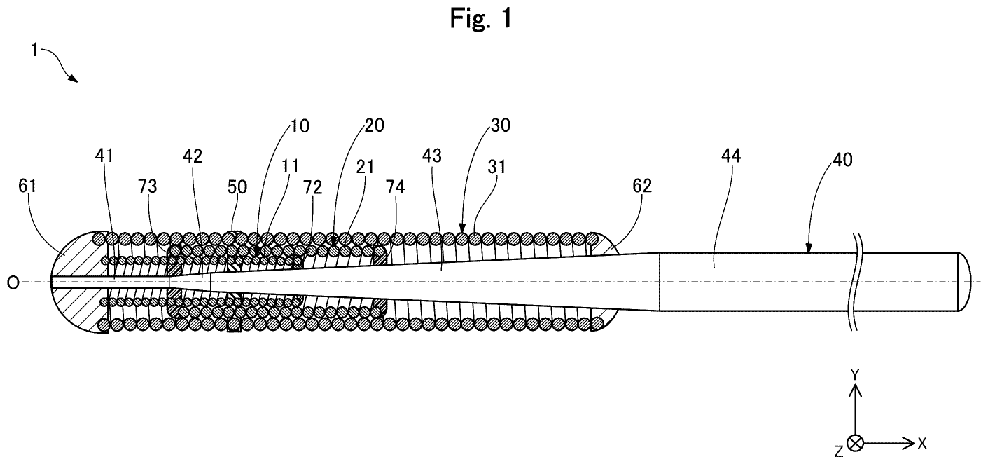

FIG. 1 is an explanatory view illustrating an example of a configuration of a guidewire 1 as a medical device. FIG. 1 illustrates a longitudinal cross-sectional configuration of the guidewire 1. The guidewire 1 is a medical device to be inserted into a blood vessel or the like. The guidewire 1 includes a first inner coil 10, a second inner coil 20, an outer coil 30, a core shaft 40, an intermediate fixing member 50, a tip end fixing member 61, a base end fixing member 62, a first base end fixing member 72, a second tip end fixing member 73, and a second base end fixing member 74. Note that the guidewire 1 can be inserted and used in a body lumen such as the one in the lymphatic system, biliary system, urinary system, respiratory tract system, digestive system, secretory gland, or reproductive organ, without being limited to the vascular system.

In FIG. 1, an axis passing through the center of the guidewire 1 is represented by an axis line O (dotted line). In the example of FIG. 1, the axis line O coincides with the axis passing through the centers of the first inner coil 10, the second inner coil 20, the outer coil 30, and the core shaft 40. However, axis line O may be different from the central axis of each of the above-mentioned constituent members. FIG. 1 illustrates mutually perpendicular X, Y, and Z axes. The X-axis corresponds to the longitudinal direction of the guidewire 1, the Y-axis corresponds to the height direction of the guidewire 1, and the Z-axis corresponds to the width direction of the guidewire 1. The left side of FIG. 1 (−X axis direction) is referred to as a “tip side” of the guidewire 1 and each constituent member, and the right side of FIG. 1 (+X axis direction) is referred to as a “base side” of the guidewire 1 and each constituent member. Further, of the two ends in the longitudinal direction (X axis direction) of the guidewire 1 and each constituent member, the end located on the tip side is referred to as a “tip end” and the other end located on the base side is referred to as a “base end”. Further, the tip end and its vicinity are referred to as a “tip portion”, and the base end and its vicinity are referred to as a “base portion”. The tip side is inserted into the living body, and the base side is operated by an operator such as a doctor. These points are the same in FIG. 1 and subsequent drawings.

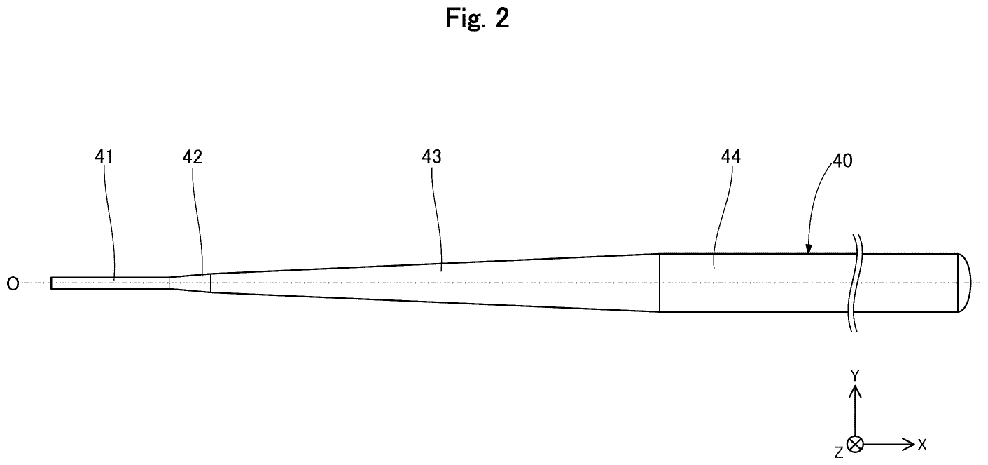

FIG. 2 is an explanatory view illustrating an example of a configuration of the core shaft 40. As illustrated in FIG. 2, the core shaft 40 has an elongated outer shape extending along the axis line O. From the tip end to the base end, the core shaft 40 includes a small diameter portion 41, a first tapered portion 42, a second tapered portion 43, and a large diameter portion 44. The core shaft 40 corresponds to a “shaft for a medical device”.

The small diameter portion 41 is disposed on the most tip side of the core shaft 40. The small diameter portion 41 is an elongated portion that extends coaxially with the axis line O of the guidewire 1 (FIG. 1 and FIG. 2) and is a cylindrical portion having a circular cross section. The tip end of the small diameter portion 41 is fixed to the first inner coil 10 and the outer coil 30 by the tip end fixing member 61. The base end of the small diameter portion 41 is connected to the first tapered portion 42. Note that the outer diameter, length in the axis line O direction, and cross-sectional shape of the small diameter portion 41 can be freely determined. For example, the small diameter portion 41 may be a portion formed in a flat plate shape having a rectangular or elliptical cross section. In this case, the small diameter portion 41 can also be referred to as a “flat plate portion”. Note that the small diameter portion 41 does not have to be coaxial with the first tapered portion 42 and the large diameter portion 44. In this case, one side surface of the small diameter portion 41 on the base side and one side surface of the first tapered portion 42 on the tip side may be joined together.

The first tapered portion 42 is disposed between the small diameter portion 41 and the second tapered portion 43. The first tapered portion 42 is substantially a truncated cone-shaped portion whose outer diameter is narrowed from the base end to the tip end. As illustrated in FIG. 2, the tip end of the first tapered portion 42 is connected to the small diameter portion 41, and the base end of the first tapered portion 42 is connected to the second tapered portion 43. Note that the outer diameter, length in the axis line O direction, and cross-sectional shape of the first tapered portion 42 can be freely determined.

The second tapered portion 43 is disposed between the first tapered portion 42 and the large diameter portion 44. The second tapered portion 43 is substantially a truncated cone-shaped portion whose outer diameter is narrowed from the base end to the tip end. As illustrated in FIG. 2, the tip end of the second tapered portion 43 is connected to the first tapered portion 42, and the base end of the second tapered portion 43 is connected to the large diameter portion 44. Note that the outer diameter, length in the axis line O direction, and cross-sectional shape of the second tapered portion 43 can be freely determined. In the illustrated example, the second tapered portion 43 has a longer length in the axis line O direction than the first tapered portion 42, and a smaller taper angle than the first tapered portion 42.

The large diameter portion 44 is disposed on the most base side of the core shaft 40. The large diameter portion 44 is substantially a cylindrical portion having a substantially constant outer diameter from the base end to the tip end. The outer diameter of the large diameter portion 44 is the same as that of the largest diameter part of the second tapered portion 43. Note that, in the present embodiment, the “same” means “approximately the same”, allowing a difference due to a manufacturing error or the like. As illustrated in FIG. 2, the tip end of the large diameter portion 44 is connected to the second tapered portion 43. The base portion of the large diameter portion 44 is held and operated by the operator. Note that the outer diameter, length in the axis line O direction, and cross-sectional shape of the large diameter portion 44 can be freely determined.

As illustrated in FIG. 1, the first inner coil 10 covers the tip portion of the core shaft 40. Specifically, the first inner coil 10 is disposed so as to surround a part of the base side of the small diameter portion 41, the first tapered portion 42, and a part of the tip side of the second tapered portion 43. The tip end of the first inner coil 10 is fixed to the core shaft 40 and the outer coil 30 by the tip end fixing member 61. The base end of the first inner coil 10 is fixed to the core shaft 40 by the first base end fixing member 72. Note that the coil average diameter of the first inner coil 10 (the average diameter of the outer diameter and the inner diameter of the first inner coil 10) and the length of the first inner coil 10 can be freely determined.

The first inner coil 10 of the present embodiment is a multi-stranded coil in which eight wires 11 are wound into multi strands, and has a substantially cylindrical shape with a constant outer diameter. The first inner coil 10 is a multi-stranded coil formed, for example, by closely twisting the eight wires 11 around a core metal so that they are in contact with each other, and then removing residual stress using a known heat treatment method, thereby removing the core metal. Note that any mode can be adopted for the first inner coil 10, and for example, the number of wires 11 constituting the first inner coil 10 is not limited to eight and can be freely determined. The first inner coil 10 is not limited to the multi-stranded coil, and may be a single stranded coil formed by winding one wire into a single strand, a single stranded twisted coil formed by winding a twisted wire obtained by twisting multiple wires together into a single strand, or a multi-stranded twisted coil formed by using multiple twisted wires obtained by twisting multiple wires together and winding the twisted wires into multiple strands.

The second inner coil 20 is disposed radially outward of the first inner coil 10 so as to cover a part of the base side of the first inner coil 10 and a part of the core shaft 40 (a part of the second tapered portion 43 in the illustrated example). In the axis line O direction, the tip end of the second inner coil 20 is located between the tip end and base end of the first inner coil 10. The tip end of the second inner coil 20 is fixed to the first inner coil 10 and the core shaft 40 by the second tip end fixing member 73. Further, in the axis line O direction, the base end of the second inner coil 20 is located closer to the base side than the base end of the first inner coil 10. The base end of the second inner coil 20 is fixed to the core shaft 40 by the second base end fixing member 74.

The second inner coil 20 is a single stranded coil formed by winding one wire 21 into a single strand. However, the second inner coil 20 is not limited to the single stranded coil, and may be a multi-stranded coil, a single stranded twisted coil, or a multi-stranded twisted coil. Note that the average coil diameter of the second inner coil 20 (average diameter of the outer diameter and the inner diameter of the second inner coil 20) and the length of the second inner coil 20 can be freely determined.

The outer coil 30 is disposed radially outward of the second inner coil 20 so as to cover the first inner coil 10, the second inner coil 20, and a part of the core shaft 40 (a part of the second tapered portion 43 in the illustrated example). In the axis line O direction, the tip end of the outer coil 30 is located at approximately the same position as the tip end of the first inner coil 10. The tip end of the outer coil 30 is fixed to the core shaft 40 and the first inner coil 10 by the tip end fixing member 61. Further, in the axis line O direction, the base end of the outer coil 30 is located closer to the base side than the base end of the second inner coil 20. The base end of the outer coil 30 is fixed to the core shaft 40 by the base end fixing member 62.

The outer coil 30 is a single stranded coil formed by winding one wire into a single strand. However, the outer coil 30 is not limited to the single stranded coil, and may be a multi-stranded coil, a single stranded twisted coil, or a multi-stranded twisted coil. Note that the average coil diameter of the outer coil 30 (average diameter of the outer diameter and the inner diameter of the outer coil 30) and the length of the outer coil 30 can be freely determined.

Note that, in the example of FIG. 1, the winding direction of the outer coil 30 is opposite to the winding direction of the first inner coil 10 and the second inner coil 20. However, the winding directions of the first inner coil 10, the second inner coil 20, and the outer coil 30 may be the same, or any one of them may be different from the other two. Further, in the example of FIG. 1, an outer peripheral surface of the first inner coil 10 and an inner peripheral surface of the second inner coil 20 are in contact with each other, and an outer peripheral surface of the second inner coil 20 and an inner peripheral surface of the outer coil 30 are in contact with each other. However, the outer peripheral surface of the first inner coil 10 and the inner peripheral surface of the second inner coil 20 may be separated from each other, and the outer peripheral surface of the second inner coil 20 and the inner peripheral surface of the outer coil 30 may be separated from each other.

The intermediate fixing member 50 is disposed between the second tip end fixing member 73 and the first base end fixing member 72 in the axis line O direction to fix a part of the first inner coil 10, a part of the second inner coil 20, a part of the outer coil 30, and a part of the core shaft 40 (the tip portion of the second tapered portion 43). The tip end fixing member 61 is disposed at the tip ends of the first inner coil 10 and the outer coil 30 to fix the tip end of the first inner coil 10 (hollow member), the tip end of the outer coil 30, and the tip end of the core shaft 40. The base end fixing member 62 is disposed at the base end of the outer coil 30 to fix the base end of the outer coil 30 and a part of the core shaft 40 (the base portion of the second tapered portion 43).

The first base end fixing member 72 is disposed at the base end of the first inner coil 10 to fix the base end of the first inner coil 10 and a part of the core shaft 40 (a part of the second tapered portion 43). The second tip end fixing member 73 is disposed at the tip end of the second inner coil 20 to fix the tip end of the second inner coil 20, a part of the first inner coil 10, and a part of the core shaft 40 (tip portion of the first tapered portion 42). The second base end fixing member 74 is disposed at the base end of the second inner coil 20 to fix the base end of the second inner coil 20 and a part of the core shaft 40 (a part of the second tapered portion 43).

The core shaft 40 is formed of a superelastic alloy (also referred to as a “pseudoelastic alloy”). Examples of the superelastic alloy include a Ni—Ti alloy and alloys of Ni—Ti and other metals. Here, the core shaft 40 of the present embodiment differs in the following points (a) and (b).
(a) Young's modulus measured by bending test (hereinafter also referred to as “first Young's modulus”).
(b) Young's modulus measured by tensile test (hereinafter also referred to as “second Young's modulus”).
   Specifically, the ratio of the first Young's modulus relative to the second Young's modulus of the core shaft 40 is 1.2 or more and 1.7 or less. That is, the ratio satisfies 1.2≤(first Young's modulus/second Young's modulus)≤1.7. Further, the ratio of the first Young's modulus relative to the second Young's modulus of the core shaft 40 is preferably 1.25 or more and 1.5 or less. That is, the ratio preferably satisfies 1.25≤(first Young's modulus/second Young's modulus)≤1.5. Further, the ratio of the first Young's modulus relative to the second Young's modulus of the core shaft 40 is particularly preferably 1.3 or more and 1.4 or less. That is, the ratio particularly preferably satisfies 1.3≤(first Young's modulus/second Young's modulus)≤1.4.

Further, the core shaft 40 can have one of the following physical properties a1 to a3:
(a1) the first Young's modulus is 46 GPa or more and 70 GPa or less (46 GPa≤first Young's modulus≤70 GPa);
(a2) the first Young's modulus is 60 GPa or more and 68 GPa or less (60 GPa≤first Young's modulus≤68 GPa); and
(a3) the first Young's modulus is 64 GPa or more and 67 GPa or less (64 GPa≤first Young's modulus≤67 GPa).

Further, the core shaft 40 can have one of the following physical properties b1 to b3:
(b1) the second Young's modulus is 30 GPa or more and 55 GPa or less (30 GPa≤second Young's modulus≤55 GPa);
(b2) the second Young's modulus is 40 GPa or more and 50 GPa or less (40 GPa≤second Young's modulus≤50 GPa); and
(b3) the second Young's modulus is 45 GPa or more and 50 GPa or less (45 GPa≤second Young's modulus≤50 GPa).

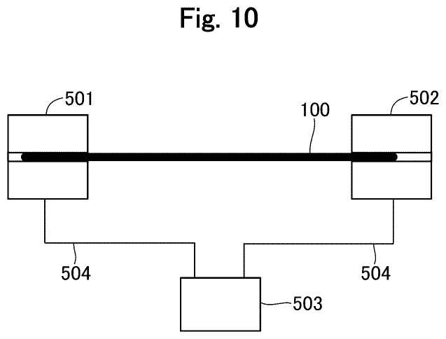

The reasons why the ratio of the first Young's modulus relative to the second Young's modulus, the first Young's modulus and the second Young's modulus are set as described above in the core shaft 40 are described below. The core shaft 40 having such anisotropy can be produced by applying shaping processing to each of the above-mentioned portions (the small diameter portion 41, the first tapered portion 42, the second tapered portion 43, and the large diameter portion 44) of the shaft for a medical device produced by a method described in FIG. 10. The anisotropy in the present embodiment includes a property in which the Young's modulus differs between tensile and bending.

FIG. 10 is a diagram for describing a method for producing the shaft for a medical device. First, a worker prepares a shaft 100 that is a material for the shaft for a medical device. The shaft 100 is a wire formed of a superelastic alloy (e.g., a Ni—Ti alloy or alloys of Ni—Ti and other metals), and has a circular cross section over the entire length from the tip end to the base end. The worker fixes one end of the shaft 100 to a first chuck 501 and fixes the other end of the shaft 100 to a second chuck 502. The worker connects a pair of conductive wires 504 connected to a power supply 503 to the first chuck 501 and the second chuck 502. The worker sets the power supply 503 to a predetermined current value, and then passes a current through the shaft 100. As a result, the shaft 100 is heated by the passing current, and at least a part of the residual stress of the shaft 100 is removed so that the shaft 100 is formed into a “shaft for a medical device” having anisotropy. Note that the method of heating by passing an electric current is merely an example of the method for producing the shaft for a medical device, and the heat treatment of the shaft 100 is not limited to the one performed by passing an electric current.

That is, the first Young's modulus value, the second Young's modulus value, and the ratio of the first Young's modulus relative to the second Young's modulus of the shaft for a medical device are adjusted by the heat treatment. The same applies to the core shaft 40 obtained by applying the shaping processing to the shaft for a medical device. Note that the first Young's modulus and the second Young's modulus of the shaft for a medical device change depending on a removal degree (degree of removal) of the residual stress described above. The worker can change the removal degree of the residual stress by adjusting the current value of the current applied to the shaft 100, and thereby keep the first Young's modulus value, the second Young's modulus value, and the ratio of the first Young's modulus relative to the second Young's modulus of the shaft for a medical device within the above-mentioned ranges.

The wire 11 constituting the first inner coil 10, the wire 21 constituting the second inner coil 20, and the wire 31 constituting the outer coil 30 can be formed of any material. The wire 11, the wire 21, and the wire 31 can be formed of, for example, a stainless steel alloy such as SUS304 or SUS316, a superelastic alloy such as a Ni—Ti alloy, a piano wire, a radiolucent alloy such as a nickel-chromium-based alloy or a cobalt alloy, gold, platinum, tungsten, or a radiopaque alloy such as an alloy containing these elements (e.g., a platinum-nickel alloy). The wire 11, the wire 21, and the wire 31 may be formed of the same material or different materials. The relationship in size between the outer diameters of the wire 11, the wire 21, and the wire 31 may be freely determined.

The intermediate fixing member 50, the tip end fixing member 61, the base end fixing member 62, the first base end fixing member 72, the second tip end fixing member 73, and the second base end fixing member 74 may be each formed using any bonding agent, for example, metal solder such as silver solder, gold solder, zinc, an Sn—Ag alloy, or an Au—Sn alloy, or an adhesive such as an epoxy-based adhesive.

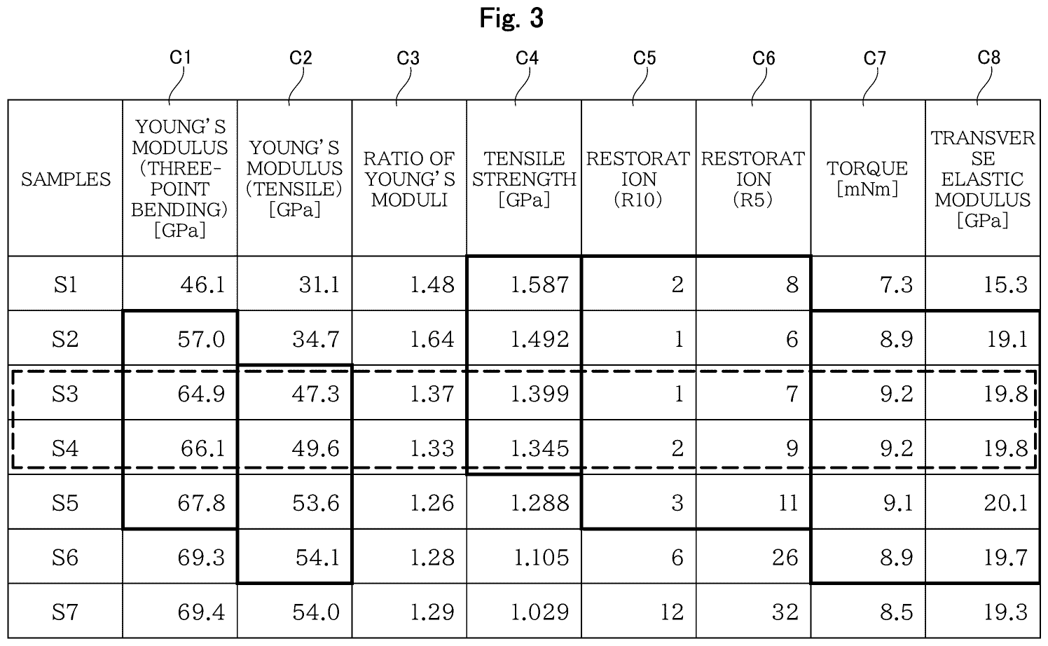

FIG. 3 is a table showing results of various tests on the shafts for medical devices. FIG. 3 shows the test results obtained by performing a three-point bending test, a tensile test, a restoration test, and a torque test on samples S1 to S7 of the shafts for medical devices. All of the samples S1 to S7 are the shafts for medical devices having anisotropy produced by the method described in FIG. 10, and are the shafts for medical devices obtained by passing the currents of different current values. Further, the samples S1 to S7 are wires before being processed into the shape of the core shaft 40 described in FIG. 2, and are wires having a circular cross section over the entire length from the tip end to the base end. The samples S1 to S7 have the same shape. A plurality of samples for each of the samples S1 to S7 are prepared and used in each test. In FIG. 3, the rows show results of the different samples, and the columns show results of the different tests. For example, in the first row, a value in column C1 and a value in column C2 represent a value obtained by performing the three-point bending test and a value obtained by performing the tensile test, respectively, on the sample S1 produced under the same conditions.

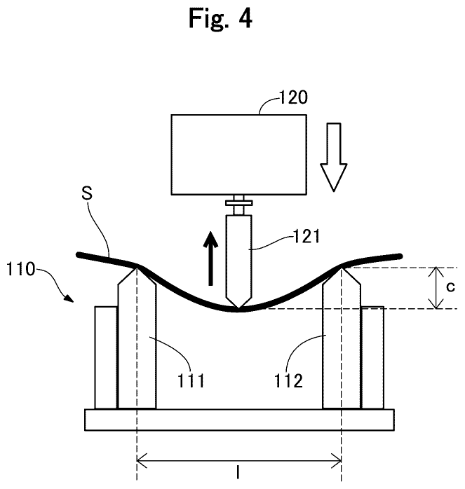

FIG. 4 is a diagram for describing a three-point bending test method. In the three-point bending test, the following procedures c1 to c4 are performed on each of the above-mentioned samples S1 to S7 using a testing machine 110 including a first support portion 111 and a second support portion 112, and a load application device 120 including a pressing portion 121 and a built-in sensor capable of measuring the load. Note that, in the following description, when any one of the samples S1 to S7 is referred to, this sample is simply referred to as “sample S” without the number. This also applies to tests other than the three-point bending test.

(c1) The sample S in a dry state (non-wet state) is supported at two points by the first support portion 111 and the second support portion 112. In this case, a linear distance 1 (fulcrum distance 1) between the fulcrum of the first support portion 111 and the fulcrum of the second support portion 112 is set to 20 mm.
(c2) The pressing portion 121 of the load application device 120 is moved in the direction of a white arrow to apply a pressing load to the sample S. In this case, a linear distance c (pressing distance c) from the fulcrums of the first support portion 111 and the second support portion 112 to a moving end point of the pressing portion 121 is set to 10 mm, and a moving speed of the pressing portion 121 is set to 5 mm/min.
(c3) In the procedure c2, a load P detected by the load application device 120 when the pressing distance is 1 mm is obtained.
(c4) A first Young's modulus E is obtained by substituting the load P obtained in the procedure c3 into the following formula (1). Note that, in addition to the obtained load P, δ_{c}=1 mm, 1-20 mm, and I=πD⁴/64 (D is the diameter of the sample S) are each substituted into the formula (1) based on the above-mentioned test conditions. The first Young's modulus obtained here is a value in a linear deformation region in a stress-strain curve (a region in which stress increases linearly with an increase in strain).

$$[ Mathematical 1 ]$$

$${δ}_{C} = \frac{{Pl}^{3}}{48 EI} \qquad ( 1 )$$

δ_{c}: Pressing distance (mm)
P: Load (N)
l: Fulcrum distance (mm)
E: First Young's modulus (GPa)
I: Second moment of area (mm⁴)

Column C1 in FIG. 3 shows the first Young's modulus E (GPa) obtained by performing the above-mentioned procedures c1 to c4 on each of the samples S1 to S7. Since bending rigidity is proportional to the Young's modulus, in the shaft for a medical device, the larger first Young's modulus can exhibit a higher support force, making it possible to improve torque transmission. From this perspective, the samples S2 to S7 are more preferable. On the other hand, in the samples S6 and S7, when the load by the pressing portion 121 is zero after completing the procedure c2, the sample S is more prone to deformation than the other samples S1 to S5. Considering the above, the samples S2 to S5 (column C1 in FIG. 3: within the bold frame) are preferable from the viewpoint of exhibiting the high support force and having resistance to deformation.

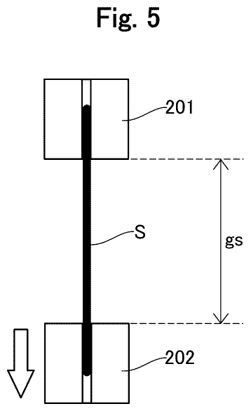

FIG. 5 is a diagram for describing a tensile test method. In the tensile test, the following procedures d1 to d3 are performed on each of the above-mentioned samples S1 to S7 using a “Shimadzu precision universal testing machine” manufactured by Shimadzu Corporation.

(d1) One end of the sample S is held by the first chuck 201 of the testing machine, and the other end of the sample S is held by the second chuck 202. In this case, a linear distance gs (grip span gs) between the first chuck 201 and the second chuck 202 is set to 100 mm. The sample S is in a dry state (non-wet state).
(d2) The grip span gs is increased at a constant speed to elongate the sample S, thereby measuring the second Young's modulus (GPa). Note that the second Young's modulus measured here is a value in the linear deformation region in the stress-strain curve.
(d3) Further, the elongation of the sample S is continued until the sample S breaks to obtain the tensile strength (GPa) of the sample S at break. The tensile strength is a value obtained by dividing the force at which the sample S breaks by the cross-sectional area of the sample S before tension (i.e., the nominal stress at break). Note that the tensile speed in the procedures d2 and d3 is set to 5 mm/min.

Column C2 in FIG. 3 shows the second Young's modulus (GPa) obtained by performing the above-mentioned procedures d1 and d2 on each of the samples S1 to S7. Since the bending rigidity is proportional to the Young's modulus, in the shaft for a medical device, the greater second Young's modulus can exhibit a higher support force. From this perspective, the samples S3 to S7 are more preferable. On the other hand, the fracture strain of the sample S7 when the sample S7 breaks in the procedure c3 is larger than that of the other samples S1 to S6. Considering the above, the samples S3 to S6 (column C2 in FIG. 3: within the bold frame) are preferable from the viewpoint of exhibiting the high support force and not causing a significant change in material properties.

Column C4 in FIG. 3 shows the tensile strength (GPa) obtained by performing the above-mentioned procedures d1 and d3 on each of the samples S1 to S7. As illustrated in FIG. 1, when the present invention is used as the core shaft 40 of the guidewire 1, the tensile strength of the shaft for a medical device is preferably high from the viewpoint of improving safety. For this reason, the samples S1 to S4 (column C4 in FIG. 3: within the bold frame) are preferable.

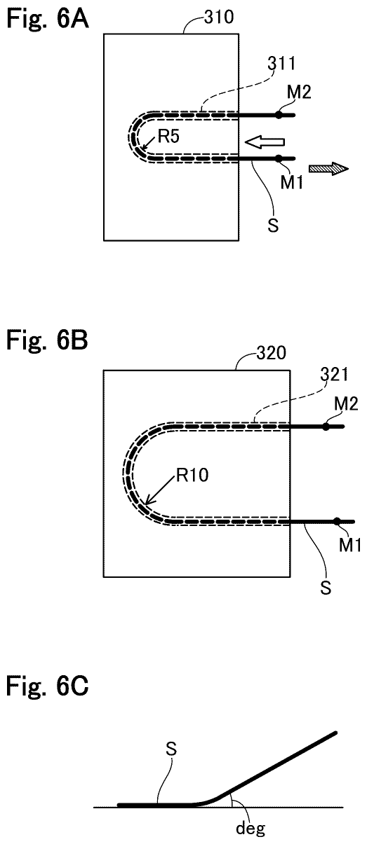

FIGS. 6A to 6C are diagrams for describing a restoration test method. FIG. 6A illustrates an example of a first jig 310. FIG. 6B illustrates an example of a second jig 320. FIG. 6C illustrates a method for measuring an angle. In the restoration test, the following procedures e1 to e3 are performed on each of the above-mentioned samples S1 to S7 using the first jig 310 in which a groove 311 with a curvature radius R of 5 mm is formed and the second jig 320 in which a groove 321 with a curvature radius R of 10 mm is formed. In the following, a case of using the first jig 310 is described as an example.

(e1) Marks M1 and M2 are placed on the sample S in a dry state (non-wet state) at positions 10 mm from both end portions. Note that the total length of the sample S is 100 mm.
(e2) The sample S is inserted into the groove 311 of the first jig 310, and the sample S is pushed in the direction of a white arrow until the mark M1 on the proximal side is located at the end portion of the first jig 310 (entrance of the groove 311). After that, the sample S is pulled in the direction of a hatched arrow until the mark M2 on the distal side is located at the end portion of the first jig 310 (exit of the groove 311). That is, in the procedure e2, the sample S is moved back and forth once inside the groove 311.
(e3) The sample S is removed from the first jig 310. As illustrated in FIG. 6C, the sample S has a bending tendency. One end of the sample S is placed on the floor, and an angle (deg) formed by the floor and the sample S is measured.

Column C5 in FIG. 3 shows the restoration (deg) obtained by performing the above-mentioned procedures e1 to e3 using the second jig 320 on each of the samples S1 to S7. Column C6 in FIG. 3 shows the restoration (deg) obtained by performing the above-mentioned procedures e1 to e3 using the first jig 310 on each of the samples S1 to S7. As illustrated in FIG. 1, when the present invention is used as the core shaft 40 of the guidewire 1, from the viewpoint of improving the resistance to deformation, the residual angle of the shaft for a medical device, representing the restoration, is preferably small. For this reason, the samples S1 to S5 (columns C5 and C6 in FIG. 3: within the bold frame) are preferable.

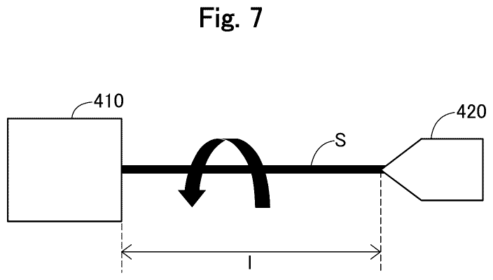

FIG. 7 is a diagram for describing a torque test method. In the torque test, the following procedures f1 to f3 are performed on each of the above-mentioned samples S1 to S7 using a tip side load cell 410 with a built-in sensor capable of measuring torque, and a chuck 420.

(f1) One end of the sample S is held by the tip side load cell 410, and the other end of the sample S is held by the chuck 420. In this case, a linear distance 1 (chuck length 1) between the chuck 420 and the tip side load cell 410 is set to 1000 mm. The sample S is in a linear (non-curved) shape and in a dry state (non-wet state).
(f2) A torque T (mNm) of the sample S on the distal side is measured by the tip side load cell 410 when the proximal side of the sample S held by the chuck 420 is rotated 720 degrees. In this case, the rotation direction is clockwise (CW), and the rotation speed is set to 3 rpm.
(f3) A transverse elastic modulus G is obtained by substituting the torque T obtained in the procedure f2 into the following formula (2). Note that, in addition to the obtained torque T, Iₚ=πD⁴/32 (D is the diameter of the sample S), φ=4π (a value obtained by converting 720 degrees into a unit rad), and 1=1000 mm are each substituted into the formula (2) based on the above-mentioned test conditions.

$$[ Mathematical 2 ]$$

$$T = {GI}_{p} \times \frac{φ}{l} \qquad ( 2 )$$

T: Torque (mNm)
G: Transverse elastic modulus (GPa)
Iₚ: Second polar moment of area (mm⁴)
φ: Twist angle (rad)
l: Chuck length (mm)

Column C7 in FIG. 3 shows the torque T (mNm) obtained by performing the above-mentioned procedures f1 and f2 on each of the samples S1 to S7. Column C8 in FIG. 3 shows the transverse elastic modulus G (GPa) obtained by performing the above-mentioned procedures f1 to f3 on each of the samples S1 to S7. For the shaft for a medical device, the greater transverse elastic modulus G can exhibit the higher torque transmission, thus the samples S2 to S6 (columns C7 and C8 in FIG. 3: within the bold frame) are preferable.

Column C3 in FIG. 3 shows the ratio of the first Young's modulus measured by the bending test relative to the second Young's modulus measured by the tensile test (i.e., the value of the first Young's modulus/the second Young's modulus). As shown in column C3, the ratio of the first Young's modulus relative to the second Young's modulus of the shaft for a medical device of the present embodiment is 1.2 or more and 1.7 or less. That is, the ratio satisfies 1.2≤(first Young's modulus/second Young's modulus)≤1.7. Further, from the results of the above-mentioned three-point bending test, tensile test, restoration test, and torque test, it is found that the samples S3 and S4 (within the dashed frame in FIG. 3) provide the best balance of the effects of improved torque transmission, improved support force, resistance to deformation, and improved tensile strength (improved safety). In other words, the ratio of the first Young's modulus relative to the second Young's modulus of the shaft for a medical device is particularly preferably 1.3 or more and 1.4 or less. That is, the ratio particularly preferably satisfies 1.3≤(first Young's modulus/second Young's modulus)≤1.4.

Further, from the results of the above-mentioned three-point bending test, it is found that, in the shaft for a medical device of the present embodiment, (a1) the first Young's modulus is 46 GPa or more and 70 GPa or less, (a2) the first Young's modulus is preferably 60 GPa or more and 68 GPa or less, and (a3) the first Young's modulus is particularly preferably 64 GPa or more and 67 GPa or less.

Further, from the results of the above-mentioned tensile tests, it is found that, in the shaft for a medical device of the present embodiment, (b1) the second Young's modulus is 30 GPa or more and 55 GPa or less, (b2) the second Young's modulus is preferably 40 GPa or more and 50 GPa or less, and (b3) the second Young's modulus is particularly preferably 45 GPa or more and 50 GPa or less.

As described above, in the shafts for medical devices of the first embodiment (samples S1 to S7), the ratio of the first Young's modulus measured by the bending test relative to the second Young's modulus measured by the tensile test is 1.2 or more and 1.7 or less, making it possible to achieve a balance between the tensile strength and the torque transmission in the shafts for medical devices. In other words, according to the samples S1 to S7, it is possible to provide the shaft for a medical device with improved tensile strength and improved torque transmission. Further, in the shafts for medical devices (samples S1, S3 to S7), the ratio of the first Young's modulus relative to the second Young's modulus is 1.25 or more and 1.5 or less, making it possible to achieve an even better balance between the tensile strength and the torque transmission. Further, in the shafts for medical devices (samples S3 and S4), the ratio of the first Young's modulus relative to the second Young's modulus is 1.3 or more and 1.4 or less, making it possible to provide the shafts for medical devices with further improved tensile strength and further improved torque transmission while achieving a balance between the tensile strength and the torque transmission.

Further, in the shafts for medical devices of the first embodiment (samples S2 to S7), the first Young's modulus is 46 GPa or more and 70 GPa or less, making it possible to improve the torque transmission of the shafts for medical devices. Further, in the shafts for medical devices (samples S3 to S5), the first Young's modulus is 60 GPa or more and 68 GPa or less, making it possible to further improve the torque transmission of the shafts for medical devices. Further, in the shafts for medical devices (samples S3 and S4), the first Young's modulus is 64 GPa or more and 67 GPa or less, making it possible to further improve the tensile strength, while further improving the torque transmission of the shafts for medical devices.

Further, in the shafts for medical devices of the first embodiment (samples S1 to S5, S7), the second Young's modulus is 30 GPa or more and 55 GPa or less, making it possible to improve the tensile strength of the shafts for medical devices. Further, in the shafts for medical devices (samples S3 and S4), the second Young's modulus is 40 GPa or more and 50 GPa or less, making it possible to further improve the tensile strength of the shafts for medical devices. Further, in the shafts for medical devices (samples S3 and S4), the second Young's modulus is 45 GPa or more and 50 GPa or less, making it possible to further improve the torque transmission, while further improving the tensile strength of the shafts for medical devices.

Further, the shaft for a medical device of the first embodiment can be configured to have superelasticity. Further, as shown in FIG. 1, when the above-mentioned shaft for a medical device is used as the core shaft 40, it is possible to provide the guidewire 1 (medical device) with improved tensile strength and improved torque transmission.

### Second Embodiment

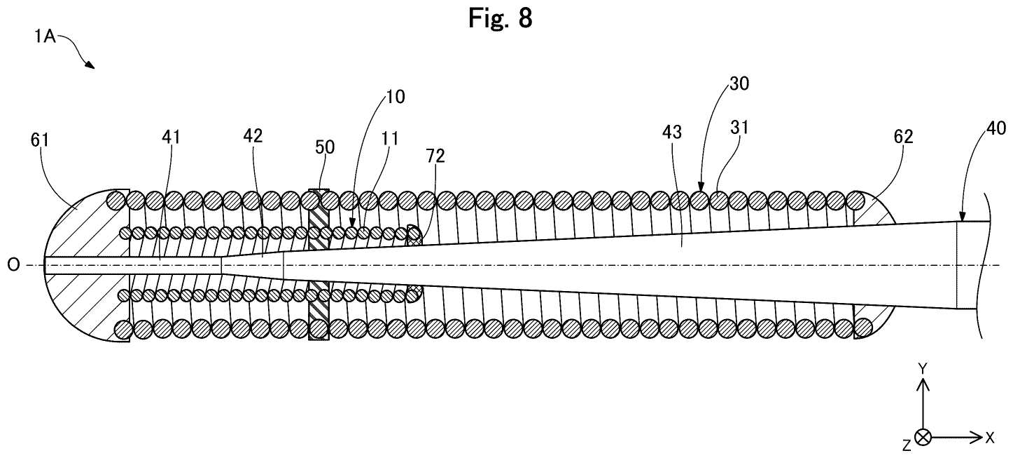

FIG. 8 is an enlarged view of a part of the tip side of a guidewire 1A of a second embodiment. The guidewire 1A of the second embodiment does not include the second inner coil 20 in the configuration described in the first embodiment. Further, the guidewire 1A does not include the second tip end fixing member 73 and the second base end fixing member 74 for fixing the second inner coil 20.

As described above, the configuration of the guidewire 1A can be modified in various manners, and the guidewire 1A may be configured by omitting the second inner coil 20. In the example of FIG. 8, the first inner coil 10 and the outer coil 30 are disposed apart from each other in the circumferential direction of the guidewire 1A. However, an outer peripheral surface of the first inner coil 10 and an inner peripheral surface of the outer coil 30 may be in contact with each other.

Further, in the configuration of FIG. 8, the guidewire 1A may be configured by omitting the first inner coil 10 and the first base end fixing member 72, and the intermediate fixing member 50, instead of omitting the second inner coil 20, or in addition to omitting the second inner coil 20. With such a guidewire 1A of the second embodiment, the same effects as those of the first embodiment described above can be achieved.

### Third Embodiment

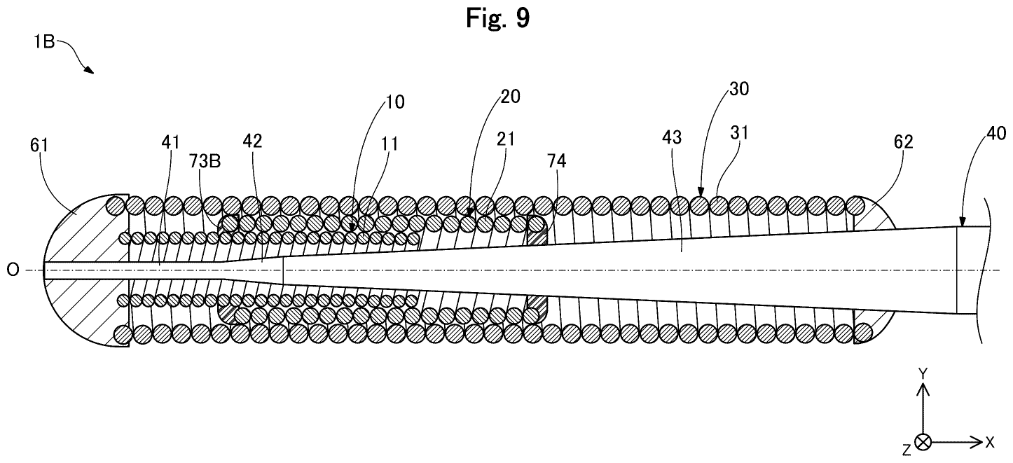

FIG. 9 is an enlarged view of a part of the tip side of a guidewire 1B of a third embodiment. The guidewire 1B of the third embodiment does not include the first base end fixing member 72 and the intermediate fixing member 50 in the configuration described in the first embodiment but includes a second tip end fixing member 73B instead of the second tip end fixing member 73.

In the guidewire 1B, the base end of the first inner coil 10 is not fixed to the core shaft 40. Further, the second tip end fixing member 73B fixes the tip end of the second inner coil 20 and a part of the first inner coil 10, but the second tip end fixing member 73B is not joined to the core shaft 40.

As described above, the configuration of the guidewire 1B can be modified in various manners. The guidewire 1B may be configured by omitting the first base end fixing member 72 and the intermediate fixing member 50. Further, the second tip end fixing member 73B may not be joined to the core shaft 40. Note that FIG. 9 is merely an example, and the second tip end fixing member 73B and the second base end fixing member 74 may be omitted instead of omitting the first base end fixing member 72, or in addition to omitting the first base end fixing member 72. Further, the intermediate fixing member 50 may or may not be omitted. With such a guidewire 1B of the third embodiment, the same effects as those of the first embodiment described above can be achieved.

### Modifications of Present Embodiment

The present invention is not limited to the above-mentioned embodiments, and can be implemented in various aspects without departing from the spirit of the invention. For example, the following modifications are also possible.

### Modification 1

In the above-mentioned first to third embodiments, examples of the configurations of the guidewires 1, 1A, and 1B each including the core shaft 40 as the shaft for a medical device are shown. However, the shaft for a medical device may be used as a constituent member of a medical device (e.g., a catheter, etc.) different from the guidewire 1. Further, FIG. 2 illustrates an example of the shape of the shaft for a medical device when the shaft for a medical device is used as the core shaft 40 of the guidewire 1. However, the shape of the shaft for a medical device may be freely changed. Specifically, the shaft for a medical device may not include at least some of the small diameter portion 41, the first tapered portion 42, the second tapered portion 43, and the large diameter portion 44 described in FIG. 2.

### Modification 2

The above-mentioned first to third embodiments show examples of the configurations of the first inner coil 10, the second inner coil 20, and the outer coil 30. However, these configurations can be modified in various manners. For example, the outer coil 30 may be configured to cover the entire core shaft 40, not just a part of the tip side of the core shaft 40. In other words, the base end of the outer coil 30 may extend to the base end of the core shaft 40. Further, for example, one or more of the first inner coil 10, the second inner coil 20, and the outer coil 30 may be a tube having a substantially cylindrical shape, instead of a coil formed by a wire wound in a spiral shape. In this case, the tube may include a slit penetrating the inside and outside of the tube or may be formed in a tubular shape without a slit. Further, a reinforcing member formed by a wire woven in a mesh pattern or a coil-shaped reinforcing member may be embedded in the tube.

### Modification 3

In the above-mentioned first to third embodiments, an example of the materials constituting each member is shown. However, each member may be formed of a material different from the above-mentioned materials. For example, the core shaft 40 may be formed of a material that is more easily plastically deformed than a superelastic alloy. Examples of the material that is more easily plastically deformed than a superelastic alloy include a stainless steel alloy such as SUS304 or SUS316. Even in this case, the core shaft 40 has the ratio of the first Young's modulus relative to the second Young's modulus described in the above-mentioned embodiments, the physical properties described in a1 to a3, and the physical properties described in b1 to b3.

### Modification 4

The configurations of the guidewires of the first to third embodiments and the configurations of the guidewires of the above-mentioned modifications 1 to 3 may be appropriately combined. The first Young's modulus may be 50 GPa or more and 69 GPa or less. The second Young's modulus may be 32 GPa or more and 54 GPa or less. The tensile strength may be 1 GPa or more and 1.6 GPa or less. The tensile strength may be 1.1 GPa or more and 1.59 GPa or less. The tensile strength may be 1.2 GPa or more and 1.5 GPa or less. The tensile strength may be 1.3 GPa or more and 1.4 GPa or less. The restoration of R10 may be 1 deg or more and 12 deg or less. The restoration of R10 may be 2 deg or more and 6 deg or less. The restoration of R5 may be 8 deg or more and 32 deg or less. The restoration of R5 may be 7 deg or more and 9 deg or less. The torque may be 7 mNm or more and 10 mNm or less. The torque may be 8 mNm or more and 10 mNm or less. The transverse elastic modulus may be 15 GPa or more and 21 GPa or less. The transverse elastic modulus may be 19 GPa or more and 21 GPa or less.

Although the present aspects have been described above based on the embodiments and modifications, the above-mentioned embodiments are for the purpose of facilitating understanding of the present aspects and do not limit the present aspects. The present aspects can be modified or improved without departing from the spirit thereof and the scope of claims, and the present aspects include any equivalents thereto. Further, the technical features of the present aspects, if not indicated as essential in the present specification, may be appropriately deleted.

### <Other Modifications or Variations

The shaft for a medical device described in connection with any of the above embodiments or modifications can further have one or more of the following attributes:

(1) The shaft can have a ratio of a first Young's modulus measured by a bending test relative to a second Young's modulus measured by a tensile test of 1.2 or more and 1.7 or less.

According to this configuration, the shaft for a medical device has the ratio of the first Young's modulus measured by the bending test relative to the second Young's modulus measured by the tensile test of 1.2 or more and 1.7 or less, making it possible to achieve a balance between tensile strength and torque transmission in the shaft for a medical device. In other words, according to the present configuration, it is possible to provide the shaft for a medical device with improved tensile strength and improved torque transmission.

(2) The shaft can have a first Young's modulus that is 4.6 GPa or more and 70 GPa or less.

(3) The can have a first Young's modulus that is 60 GPa or more and 68 GPa or less.

(4) The shaft can have a first e Young's modulus that is 64 GPa or more and 67 GPa or less.

(5) The shaft can have a second Young's modulus that is 30 GPa or more and 55 GPa or less.

(6) The shaft can have a second Young's modulus that is 40 GPa or more and 50 GPa or less.

(7) The shaft can have a second Young's modulus that is 45 GPa or more and 50 GPa or less.

(8) The shaft can have a ratio of the first Young's modulus relative to the second Young's modulus that is 1.25 or more and 1.5 or less.

(9) The shaft can have a ratio of the first Young's modulus relative to the second Young's modulus that is 1.3 or more and 1.4 or less.

(10) The shaft can have superelasticity.

(11) The shaft can have a circular cross section.

(12) The shaft can have be subjected to a heat treatment that was effective to adjust the ratio of the first Young's modulus relative to the second Young's modulus.

(13) According to another aspect, a medical device is provided that can include any of the shafts or variations thereof described above.

### DESCRIPTION OF REFERENCE NUMERALS

1, 1A, 1B Guidewire
10 First inner coil
11, 21, 31 Wire
20 Second inner coil
30 Outer coil
40 Core shaft
41 Small diameter portion
42 First tapered portion
43 Second tapered portion
44 Large diameter portion
50 Intermediate fixing member
61 Tip end fixing member
62 Base end fixing member
72 First base end fixing member
73, 73B Second tip end fixing member
74 Second base end fixing member
100 Shaft
110 Testing machine
111 First support portion
112 Second support portion
120 Load application device
121 Pressing portion
201 First chuck
202 Second chuck
310 First jig
311, 321 Groove
320 Second jig
410 Tip side load cell
420 Chuck
501 First chuck
502 Second chuck
503 Power supply
504 Conductive wire

What is claimed is:

1. A shaft for a medical device, wherein
the shaft has a ratio of a first Young's modulus measured by a bending test relative to a second Young's modulus measured by a tensile test is 1.2 or more and 1.7 or less.

2. The shaft according to claim 1, wherein
the first Young's modulus is 46 GPa or more and 70 GPa or less.

3. The shaft according to claim 2, wherein
the first Young's modulus is 60 GPa or more and 68 GPa or less.

4. The shaft according to claim 3, wherein
the first Young's modulus is 64 GPa or more and 67 GPa or less.

5. The shaft according to claim 1, wherein
the second Young's modulus is 30 GPa or more and 55 GPa or less.

6. The shaft according to claim 5, wherein
the second Young's modulus is 40 GPa or more and 50 GPa or less.

7. The shaft according to claim 6, wherein
the second Young's modulus is 45 GPa or more and 50 GPa or less.

8. The shaft according to claim 1, wherein
the ratio of the first Young's modulus relative to the second Young's modulus is 1.25 or more and 1.5 or less.

9. The shaft according to claim 8, wherein
the ratio of the first Young's modulus relative to the second Young's modulus is 1.3 or more and 1.4 or less.

10. The shaft according to claim 1, wherein
the shaft has superelasticity.

11. The shaft according to claim 1, wherein
the shaft has a circular cross section.

12. The shaft according to claim 1, wherein
the shaft has been subjected to a heat treatment that was effective to adjust the ratio of the first Young's modulus relative to the second Young's modulus.

13. The shaft according to claim 2, wherein the second Young's modulus is 30 GPa or more and 55 GPa or less.

14. The shaft according to claim 13, wherein the second Young's modulus is 40 GPa or more and 50 GPa or less.

15. The shaft according to claim 14, wherein the second Young's modulus is 45 GPa or more and 50 GPa or less.

16. The shaft according to claim 2, wherein the ratio of the first Young's modulus relative to the second Young's modulus is 1.25 or more and 1.5 or less.

17. The shaft according to claim 16, wherein the ratio of the first Young's modulus relative to the second Young's modulus is 1.3 or more and 1.4 or less.

18. A medical device comprising the shaft according to claim 1.
